(19) United States
(12) Patent Application Publication
(10) Pub. No.: US 2005/0148007 A1
(43) Pub. Date: Jul. 7, 2005

(54) Bioluminescent assays and cells useful therein

(75) Inventors: Jiri Aubrecht, Stonington, CT (US); Jeffery J. Osowski, Preston, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/020,878

(22) Filed: Dec. 22, 2004

Related U.S. Application Data
(60) Provisional application No. 60/532,528, filed on Dec. 24, 2003.

(51) Int. Cl. C12Q 1/68; C12Q 1/18; C12N 1/18; C12N 15/74

(52) U.S. Cl. 435/6; 435/32; 435/254.21; 435/483

(57) ABSTRACT

The present invention relates to novel bioluminescent assays and to cells and kits useful therein.

### CROSS REFERENCE

This patent application claims the benefit of U.S. Provisional Patent Application No. 60,532,528, filed on Dec. 24, 2003 and incorporated herein by reference in its entirety.

### FIELD OF THE INVENTION

The present invention relates to novel bioluminescent assays and to cells and kits useful therein.

### BACKGROUND OF THE INVENTION

It is generally accepted that the mutagenic potential of a chemical agent is roughly proportional to the agent's carcinogenic potential. Grossblatt, N. (1983). An early determination of whether a particular agent presents a hazard of mutagenicity is fundamental to the development of products for the chemical, cosmetic, food additive and pharmaceutical industries.

Mutagens are agents that cause an increase in the rate of mutation, i.e. detectable and heritable structural changes in the genetic material of an organism. Such changes may include the addition or deletion of a whole chromosome, a structural change to a chromosomes (e.g., a translocation) and a structural change to a portion of the genomic sequence (e.g., point mutations, mutations to multiple sequential nucleotides and deletions of portions of the genomic sequence). Because mutagentic changes can damage or otherwise interfere with the action of genes, mutagens are characterized as genotoxins, i.e. agents that are toxic to genes.

A widely known in vitro test for detecting mutagens is the Ames Assay. This test measures the ability of an agent to cause a reversal of mutations in histidine dependent tester strains of Salmonella typhimurium, thereby restoring the cells' ability to make their own histidine (Ames et al., 1973a, 1973b, and 1975; see also, Ames, B. N., 1971). Methods similar to the Ames Assay have been developed to measure the restoration of ampicillin resistance by a reverse mutation of the beta-lactamase gene in strains of Salmonella (Lee, C-C, et al., 1994, and Hour, T-C., et al., 1995) and in strains of Escherichia coli (Bosworth, D. et al., 1987; and Foster, P. L. et al., 1987). All such tests employ bacterial strains that detect mutagens by a single nucleotide reverse mutation; either, by substitution of one nucleotide for another or by a nucleotide insertion or deletion causing a sequence frameshift.

Modifications of the Ames-type assays have been reported, for example, in Yahagi, T. et al. (1975); Prival, M. J. and Mitchell, V. D. (1982); Haworth, S. et al. (1983); Kado, N. Y., et al. (1983); and Reid, T. M., et al. (1984); and Current Protocols Mutagenesis and Adduct Formation, Chapter 3 Introduction, Unit 3.1 The Salmonella (Ames) Test for Mutagenicity, Alternate Protocol 1: Plate Assay With Preincubation Procedure; Alternate Protocol 2: Desiccator Assay for Volatile Liquids; Alternate Protocol 3: Desiccator Assay for Gases; Alternate Protocol 4: Reductive Metabolism Assay; Alternate Protocol 5: Modified (Kado) Microsuspension Assay. These modifications have generally been directed to minimizing the amounts of test agent used and increasing the speed and operational efficiency of the assay.

Co-assigned U.S. patent application Ser. No. 10/029,741 discloses novel Ames-type assays, comprising, inter alia, contacting a bacterial cell with a test agent and an exogenous metabolic activation system, where the bacterial cell comprises an expressible heterologous lux(CDABE) gene complex (or operon) and a reversible point mutation in a gene which in a non-mutated form encodes a polypeptide whose functioning is critical for the cell to be metabolically active in a selective medium.

Additional methods for identifying mutagenic agents have been described, including: the mouse lymphoma system for point mutations (Amacher et al. (1979)); the Chinese hamster ovary system for chromosome aberrations and sister chromatid exchange, (Evans (1983) and Wolff (1983)); the micronucleus assay (Fenech and Morley (1985)); and the drosophila mutagenesis assay (Rasmuson et al. (1978)).

Schiestl et al. (1988) reported a positive selection system for intrachromosomal recombination in the yeast, Saccharomyces cerevisiae, by integration of a plasmid containing an internal fragment of the HIS3 gene at the HIS3 locus resulting in two copies of the gene with terminal deletions at the 3′ end of one and 5′ end of the other.

Sommers et al. (1995) reported an automated method for the intrachromosomal recombination system described by Schiestl et al. (1988) utilizing multi-well plates and measuring the reversion frequency using a micro-well fluctuation method.

Cote et al (1995) disclose an Ames mutagenicity assay using bioluminescent strains of Salmonella typhimurium.

Although the currently available methods for evaluating mutagenic potential of test agents, particularly the Ames Test, have served a useful and important function, there nevertheless exists a need for new methods that provide a reliable and accurate assessment of potential mutagenicity by means that are relatively fast and economical.

### SUMMARY OF THE INVENTION

The present invention relates, in part, to methods for testing an agent, comprising, treating a eukaryotic cell comprising a DEL selection marker and a bioluminescent marker with a test agent and measuring the level of bioluminescence from the cell in the presence of a suitable selection medium.

Another aspect of this invention provides methods for testing an agent, comprising, providing a eukaryotic cell culture comprising a DEL selection marker and a bioluminescent marker, treating a treated portion of said eukaryotic cell culture with a test agent, measuring the number of bioluminescent cells of the treated portion; and measuring the number of bioluminescent cells of an untreated portion of the eukaryotic cell culture.

A further aspect of this invention provides methods for testing an agent, comprising, providing a eukaryotic cell culture comprising a DEL selection marker and a bioluminescent marker, treating a treated portion of said eukaryotic cell culture with a test agent; measuring the number of bioluminescent cells of the treated portion in the presence of a suitable selection medium, measuring the number of bioluminescent cells of an untreated portion of the eukaryotic cell culture in the presence of a suitable selection medium, and characterizing the test agent according to a category selected from: an agent that increases the number of bioluminescent cells of said treated portion as compared to said untreated portion in the presence of said selection medium; and an agent that does not increase the number of bioluminescent cells of said treated portion as compared to said untreated portion in the presence of said selection medium.

An additional aspect of this invention provides eukaryotic cells comprising a DEL selection marker and a bioluminescent marker.

In a preferred embodiment of the method aspects of the invention, said increase in bioluminescence is statistically significant as compared to said untreated portion.

In a further preferred embodiment of the method aspects of the invention, said increase in bioluminescent cells is at least two-fold as compared to said untreated portion.

In another preferred embodiment of the method aspects of the invention, said eukaryotic cells are derived from a cell selected from: a mammalian lymphoid cell; a human lymphoblastoid cell; a yeast cell; and a Saccharomyces cerevisiae cell, preferably a Saccharomyces cerevisiae cell.

In a preferred embodiment of the cell aspects of the invention, said eukaryotic cell is derived from a cell selected from: a mammalian lymphoid cell; a human lymphoblastoid cell; a yeast cell; and a Saccharomyces cerevisiae cell, preferably a Saccharomyces cerevisiae cell, and more preferably a Saccharomyces cerevisiae cell of the strain RS112-luc.

### BRIEF DESCRIPTION OF THE DRAWINGS

For further understanding of the invention as well as other objects and further features thereof, reference is made to the following detailed description of various preferred embodiments thereof taken in conjunction with the accompanying drawings wherein:

FIG. 1 depicts the mechanism of the DEL recombination. The DEL recombination is initiated by DNA double strand break, which is repaired by single strand annealing. This leads to the deletion of the duplicated allele with the intervening sequence (Leu) and restoring the wild-type His3 marker.

FIG. 2 is a map of the pYES-GL3-GPD plasmid showing the luciferase gene, luc+, a constitutive glyceraldehydes-3-phosphate dehydrogenase (GPD) promoter, and, from the pYES6/CT backbone vector, the bacterial and yeast origins of replication (pUC ori and 2μ, respectively), and blasticidin and ampicillin resistance genes.

FIG. 3 depicts the principle of the bioluminescent detection of cells that undergo DEL recombination. Recombination of the DEL marker renders the cells capable of surviving under selection medium. Only surviving cells are metabolically active and produce enough ATP to maintain bioluminescent phenotype.

FIG. 4 is a graphical representation of the effect of methyl methanesulfonate (MMS) treatment of RS112luc cells on DEL recombination frequency (part A) and on survival (part B).

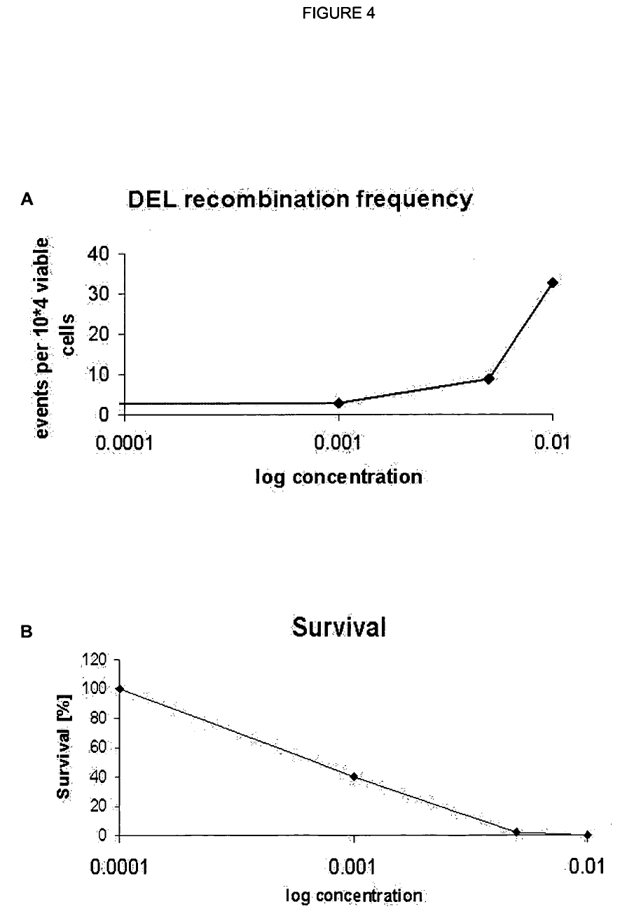

### DETAILED DESCRIPTION OF THE INVENTION

The terms used herein have their usual meaning in the art. However, to further clarify the present invention and for convenience, the meaning of certain terms and phrases employed in the specification, including the examples and appendant claims are provided below.

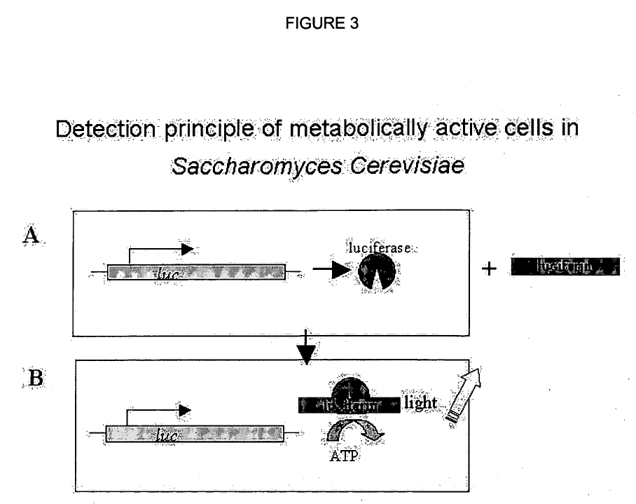

“Bioluminescence” means light emission in a living cell wherein the light emission is dependent upon and responsive to metabolic activity (see, for example, FIG. 3).

“Bioluminescent marker” means a nucleotide sequence that, when incorporated into a cell and expressed, causes bioluminescence during metabolic activity of the cell.

“Gene” means a nucleic acid fragment that expresses a specific protein, including regulatory sequences preceding (5′ non-coding sequences) and following (3′ non-coding sequences) the coding sequence. The term gene, includes endogenous genes in their natural location in the genome or foreign genes that are not normally found in the host organism, but are introduced into the host organism by gene transfer.

“Mutagens” are agents that cause an increase in the rate of mutation. Mutagens may have genotoxic effect by damaging or otherwise interfering with the action of genes.

“Mutation” is a detectable and heritable structural change in the genetic material of an organism, and may include the addition or deletion of a whole chromosome, a structural change to a chromosomes (e.g., a translocation) and a structural change to a portion of the genomic sequence (e.g., point mutations, mutations to multiple sequential nucleotides and deletions of portions of the genomic sequence).

“DEL selection marker” means a disrupted genetic sequence wherein: (1) the disruption comprises an insertion of a nucleotide sequence within the genetic sequence; (2) said nucleotide sequence comprises one duplication of a portion of the genetic sequence; (3) the head-to-tail (i.e., 5′ end to 3′ end) orientation of the duplicated portion of said nucleotide sequence is the same as that of the genetic sequence; and (4) the genetic sequence is useful for phenotypic selection of the cell when grown on suitable selection media. Various embodiments of DEL selection markers are described below.

“Selection medium” means a composition which can be used for phenotypic selection of cells. For example, a nutrient composition which lacks histidine can be used to selectively screen for yeast cells that are able to produce histidine. A nutrient composition which contains the antibiotic G418 can be used to selectively screen for cells that have the neo resistance gene.

“Suitable selection medium” when used with reference to a DEL selection marker means a selection medium having a composition that can be used for phenotypic selection of cells based upon the genetic sequence of the DEL selection marker.

The abbreviations used herein have their usual meaning in the art. However, to further clarify the present invention, for convenience, the meaning of certain abbreviations are provided as follows: “° C.” means degrees centigrade; “AL” means microliter; “ATCC” means the American Type Culture Collection located in Manassas, Va. (website at www.atcc.org); “DNA” means deoxyribonucleic acid; “EDTA” means ethylenediamine tetra-acetic acid; “g” means gram; “kg” means kilogram; “mg” means milligram; “mL” means milliliter; “mM” means millimolar; MMS means methyl methanesulfonate; “ng” means nanogram; “PBS” means phosphate buffered saline; “RNA” means ribonucleic acid; and “RPM” means revolutions per minute.

Environmental factors have been linked to the causation of cancer. In fact, the role of environmental factors may have a closer causal link to cancer than heredity (Lichtenstein, et al. (2000)). As a consequence, efforts have been made to identify and reduce human exposure to natural and man-made chemical agents that are known or suspected carcinogens.

As stated above, it has been generally accepted that the carcinogenic potential of a chemical agent can, at least in part, be predicted by its mutagenicity. Grossblatt, N. (1983). This has enabled industries such as the chemical, cosmetic, food additive and pharmaceutical industries to alleviate the carcinogenic risk of their products by minimizing their mutagenic properties.

The DEL assay, also known as the intrachromosomal recombination assay, first described by Schiestl et al. (1988) using Saccharomyces cerevisiae, measures deletions of parts of the genomic sequence that are induced in target gene sequences by mutagens. Hence, this assay enables the evaluation of test compounds for their mutagenic potential.

The target gene sequences used in the DEL assay are genes whose function has been disrupted by the integration of an exogenous DNA fragment. For example, Schiestle et al. (1988) describes the use of a strain of S. cerevisiae designated “RSY6” (available from the ATCC, deposit number 201682), in which the HIS3 gene is disrupted by the integration of an exogenous DNA fragment. The resulting his-yeast strain requires histidine in its growth medium in order to grow. In histidine-free medium, a very small number of cells will spontaneously revert to his+. However, when the cells are treated with a mutagen, the reversion rate increases beyond the normal background level.

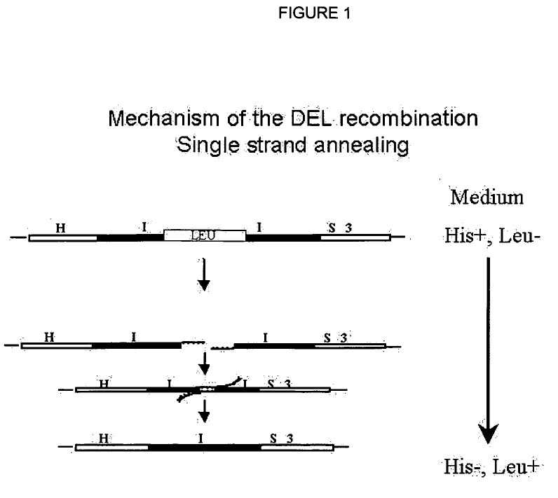

A proposed mechanism for the function of the DEL assay is illustrated in FIG. 1 (Galli and Schiestl (1998)). As FIG. 1 shows, a mutagen causes the formation of double-stranded DNA breaks. When such breaks occur in the disrupted gene, a cell's own repair mechanism may result in removal of the exogenous DNA and repair of the sequence via single-strand annealing, thus resulting in reversion of the gene to its wild-type form.

The DEL assay has certain advantages over other mutagenicity assays. For example, it has been reported that the DEL assay has better predictability of carcinogenicity than the more commonly used Salmonella reverse mutation Ames assay. Many carcinogenic compounds which give negative results using the Ames assay are positive by the DEL method. (Bishop and Schiestl (2000)).

However, one disadvantage of the currently available DEL assay is its impracticality for large scale and automated screening of potential mutagens (i.e., high throughput screening). For example, the current assay requires that cells be given enough time to grow into visible colonies in order to determine whether a test compound is a potential carcinogen. Moreover, because of the need to visualize growing colonies, the current assay cannot be miniaturized, for example, into a multi-well plate system, which would enable a reduction in the amount of test agent necessary.

The present invention is based, in part, on the discovery of DEL-type methods that use bioluminescence as a positive indicator of mutagenicity as DEL recombination events. By this invention, one may expeditiously and economically test agents of unknown carcinogenic potential for DEL recombination in a manner that was previously unavailable.

The invention involves the use of cells having, as a component, a bioluminescent marker as well as a disrupted DEL-type selection marker. The bioluminescent marker enables very early detection of cells that have reverted to the wild-type phenotype as a result of the DEL recombination. Bioluminescence in revertant cells grown in a selection medium occurs as a result of their metabolic activity (see FIG. 3), as compared to non-revertants. If sufficiently sensitive detection means are available, the methods of the invention enable detection of individual cell revertants or microcolonies of those cells very soon after treatment with test agents. This would obviate the need for allowing cells to grow into large colonies in order to allow detection.

In contrast to the currently available DEL assay, the methods and cells of the invention which are based on the detection of bioluminescence of revertant cells, enable the use of a miniaturized system for testing agents, for example, systems that use multi-well plates. The methods and cells of the invention enable a significant reduction in the amount of test agent necessary for mutagenicity testing. This can be a significant advantage where test agents are only available in small quantities. In addition, the use bioluminescence allows the use of sensitive devices such as CCD chip based photon counting cameras for fast and accurate detection of the bioluminescent signal.

As those with skill in the art will appreciate based upon the present disclosure, any suitable eukaryotic cells may be used in the practice of this invention. For example, the cells may originate from vertebrate organisms, such as mammals, birds, fishes, reptiles and amphibians as well as invertebrates (e.g., insects, nematodes) and single-celled eukaryotes. For multi-celled eukaryotes, the cells may be derived from any organ or tissue, including blood, endothelium, thymus, spleen, bone marrow, liver, kidney, heart, testis, ovary, heart and skeletal muscle, and can be primary cells or cells derived from immortalized cell lines. Preferred cells include human lymphoblastoid cell lines such as GM6804 (see, for example, Monnat, R. J. et al. (1992) and Aubrecht, J. et al. (1995)) and yeast cells, for example, of the species, Saccharomyces cerevisiae.

Cells and cell lines for use in the methods of this invention may be obtained, for example, from the ATCC, Manassas, Va. 20110-2209.

As defined above, a DEL selection marker means a disrupted genetic sequence wherein: (1) the disruption comprises an insertion of a nucleotide sequence within the genetic sequence; (2) said nucleotide sequence comprises one duplication of a portion of the genetic sequence; (3) the head-to-tail (i.e., 5′ end to 3′ end) orientation of the duplicated portion of said nucleotide sequence is the same as that of the genetic sequence; and (4) the genetic sequence is useful for phenotypic selection of the cell.

For example, where a genetic sequence comprises the elements A-B-C-D-E-F-G, suitable DEL selection markers based upon such a genetic sequence may encompass the sequences A-B-C-B-C-D-E-F-G, A-B-C-X-B-C-D-E-F-G, and A-B-C-B-C-X-D-E-F-G, wherein X is itself a selection marker that could be used to select transformed cells that have successfully incorporated the disruption.

It will be appreciated by those with skill in the art, based upon the present disclosure, that any suitable phenotype selection marker may be used for the DEL selection marker in the practice of the invention. It will be further appreciated that the type of selection marker used may, in part, depend upon the types of cells used in the practice of the invention.

In one embodiment of the invention, the DEL selection marker comprises a disruption of the function of a nutrient marker gene, such that the cell requires, as a result of this disruption, a specific nutrient in order to maintain its viability, metabolic activity or growth. In this embodiment, agents may be tested for their ability to cause reversion of the nutrient marker to its non-disrupted form, thus enabling cells to thrive in media lacking the corresponding nutrient. An exemplary nutrient markers includes the his3 in yeast cells which alters cellular requirements for histidine. Other nutrient markers will be apparent to those with skill in the art based upon the present disclosure.

In another embodiment, the DEL selection marker is a gene that conveys resistance to specific physical or chemical agents that would otherwise be toxic to the cell (i.e., hinder viability, metabolic activity or growth). Such “resistance markers” confer resistance to the cell against chemical agents, including, for example, antibiotics, antimetabolites or herbicides. A disruption of the function of the resistance marker gene causes toxicity to the cell when exposed to the toxic agent. As such, this embodiment comprises the testing of agents for their ability to cause reversion of the gene to its non-disrupted form, thereby enabling the cells to thrive in media containing the toxic substance. Exemplary resistance markers include dhfr (dihydrofolate reductase) which confers resistance to methotrexate; neo, which confers resistance to the aminoglycosides, neomycin and G418; and als and pat, which confer resistance to chlorsulfuron and phosphinotricin acetyltransferase, respectively (see, Wigler, M. et al. (1980); Colbere-Garapin, F. et al. (1981)). Other resistance markers are known to those with skill in the art or will be apparent to them based upon the present disclosure.

Those with skill in the art will appreciate, based upon this disclosure, that, within the scope of the present invention, DEL selection markers may also encompass a non-disrupted nutrient or resistance marker that is controllable by a secondary genetic element, wherein the function of the secondary genetic element is disrupted. Such secondary genetic element may include a gene which encodes a transcriptional activator protein which binds to an activation domain, thereby initiating or accelerating the rate of transcription of the nutrient or resistance marker. Hence, according to this embodiment, agents may be tested for their ability to cause reversion of the secondary genetic element to its functional form, thereby enabling the expression of the nutrient marker or resistance marker gene. An exemplary transcriptional activators and activation domain sequence combination includes the Tet-controlled transactivator which is part of the BD™ Tet-Off Gene Expression System (BD Biosciences, Palo Alto, Calif.). Other transcriptional activators and activation domain sequences are known to those with skill in the art or will apparent to them based upon the present disclosure.

As will be further appreciated by those with skill in the art based upon the present disclosure, the DEL selection markers may also encompass a non-disrupted negative selectivity marker gene that is controllable by a transcriptional repressor genetic element, wherein the function of the transcriptional repressor is disrupted. When active, the negative selectivity marker is toxic to the cell. Hence, according to such embodiments, agents may be tested for their ability to cause reversion of the transcriptional repressor to its functional form, thereby enabling the expression of the negative selectivity marker gene. An exemplary negative selectivity marker is the herpes simplex virus gene, thymidine kinase, which causes cytotoxicity in the presence of the drug, gancyclovir (Moolton (1986)). Other negative selectivity markers include Hprt (cytotoxicity in the presence of 6-thioguanine or 6-thioxanthine), and diphtheria toxin, ricin toxin, and cytosine deaminase (cytotoxicity in the presence of 5-fluorocytosine).

A transcriptional repressor genetic element would, when expressed, repress expression of the negative selectivity marker. An exemplary transcriptional repressor is through the use of RNA interference (RNAi) using methods, for example, described in Fire et al. (1998), in Brummelkamp et al. (2002) and by other methods known to those with skill in the art.

As will be apparent to those with skill in the art based upon the present disclosure, the disrupted gene or genetic element that makes up a DEL selection marker used in the methods and cells of this invention, may be an endogenous gene or genetic element or it may be an exogenous gene or genetic element introduced into a progenitor cell by recombinant methods that are well known to those with skill in the art based upon the present disclosure. Moreover, the cells used in this invention may be either haploid, having one copy of each type of chromosome, or diploid, having two copies of each chromosome-type. Hence, when diploid cells are used in the methods and cells of this invention and the disrupted gene or genetic element that makes up a DEL selection marker is an endogenous gene or genetic element, or when there is otherwise more than one copy of an endogenously existing gene or genetic element, it is preferable to disrupt all copies of said gene or genetic element for the practice of methods and use of cells of the invention.

In a preferred embodiment, the DEL selection marker for use in Saccharomyces cerevisiae yeast cells comprises a HIS3 gene which is disrupted by insertion of the plasmid pRS6 as described in Schiestl et al. (1988) and which is contained in the S. cerevisiae strains RSY6 and RS112 as described in U.S. Pat. No. 4,997,757.

It will be appreciated by those with skill in the art, based upon the present disclosure, that any suitable bioluminescent marker may be used in the practice of the invention. It will be further appreciated that the type of bioluminescent marker used may, in part, depend upon the types of cells used in the practice of the invention. An exemplary bioluminescent marker for use in yeast cells is the firefly luciferase (luc) gene (GeneBank® accession number AAA89084) driven by a constitutive glyceraldehydes-3-phosphate dehydrogenase (GPD) promoter. Mumberg, D. et al. (1995). The bioluminescence catalyzed by the luc gene requires the substrate (luciferin) and energy in the form of endogenous ATP. So long as the medium in which the cells grow contains luciferin as a supplement, the bioluminescence of yeast cells is exclusively dependent on the availability of intracellular ATP. Since the intracellular ATP concentration is dependent on energy metabolism, the bioluminescent output represents the level of metabolic activities of yeast cell. In the methods of the invention, a test compound which causes a deletion recombination event to restore function of a DEL selection marker allows the cells to maintain metabolic activities and multiply in the absence of the applicable nutrient or the presence of a potentially cytotoxic substance (see FIG. 3).

Other bioluminescent markers that may be used in the methods and cells of this invention are known to those with skill in the art or will be apparent to them based upon the present disclosure. For example, Bronstein et al. (1994) describe bioluminescent markers that may be used in this invention.

The bioluminescent markers and DEL selection markers that are used in the methods and cells of the invention may be incorporated into a cell by inserting the nucleotide sequences encoding such markers into an appropriate vector. Such vectors may be designed so that they are stably incorporated into the chromosomal DNA of a cell or they may be designed to express the applicable marker without chromosomal integration.

Expression vectors containing the necessary elements for transcriptional and translational control of the inserted coding sequence in a cell may be used to incorporate into a cell a biologically active bioluminescent marker or a DEL selection marker that will become biologically active upon reversion following treatment with a test agent. The transcriptional and translational control elements include regulatory sequences, such as enhancers, constitutive and inducible promoters, and 5′ and 3′ untranslated regions in the vector and in polynucleotide sequences encoding the applicable marker. Such elements may vary in their strength and specificity. Specific initiation signals may also be used to achieve more efficient translation of sequences encoding the markers. Such signals include the ATG initiation codon and adjacent sequences, e.g. the Kozak sequence. In cases where sequences encoding a marker and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994)).

Methods which are well known to those skilled in the art based upon the present disclosure may be used to construct expression vectors containing sequences encoding bioluminescent markers and DEL selection markers and appropriate transcriptional and translational control elements. These methods include in vitro recombinant DNA techniques, synthetic techniques, and in vivo genetic recombination. (See, e.g., Sambrook, J. et al. (1989), ch. 4, 8, and 16-17; Ausubel et al. (2003), 9, 13, and 16).

For embodiments of the invention in which the DEL selection marker involves disruption of an endogenous gene, a preferred method of incorporating a DEL selection marker is through homologous recombination. Homologous recombination methods for incorporating engineered gene constructs into the chromosomal DNA of cells are well known to those skilled in the art and/or those that will be further apparent to them based upon the present disclosure.

In the preparation of cells containing a DEL selection marker, a DEL selection marker targeting vector is introduced into a cell having the undisrupted target gene. The introduced vector targets the gene using a nucleotide sequence in the vector that is homologous to the target gene. The homologous sequence facilitates hybridization between the vector and the sequence of the target gene. Hybridization causes integration of the vector sequence into the target gene through a crossover event, resulting in disruption of the target gene.

General principles regarding the construction of vectors used for targeting are reviewed in Bradley et al. (1992). Guidance regarding the selection and use of sequences effective for homologous recombination, based on the present description, is described in the literature (see, for example, Deng and Capecchi (1992); Bollag et al. (1989); and Waldman and Liskay (1988)).

As those skilled in the art will recognize based upon the present invention, a wide variety of cloning vectors may be used as vector backbones in the construction of the DEL selection marker targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pMB9, pBR325, pKH47, pBR328, pHC79, phage Charon 28, pKB11, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mas.). However, any other vectors, e.g. plasmids, viruses, or parts thereof, may be used as long as they are replicable and viable in the desired host. The vector may also comprise sequences which enable it to replicate in a host cell whose genome is to be modified. The use of such a vector can expand the interaction period during which recombination can occur, increasing the efficiency of targeting (see Ausubel et al (2003), Unit 9.16, FIG. 9.16.1).

The specific host cell employed for propagating the targeting vectors of the present invention is not critical. Examples include E. coli K12 RR1 (Bolivar et al., (1977)), E. coli K12 HB101 (ATCC No. 33694), E coli MM21 (ATCC No. 336780), E. coli DH1 (ATCC No. 33849), E. coli strain DH5a, and E. coli STBL2. Alternatively, host cells such as C. cerevisiae or B. subtilis can be used. The above-mentioned exemplary hosts, as well as other suitable hosts are available commercially (e.g., Stratagene, La Jolla, Calif.; and Life Technologies, Rockville, Md.).

Preferably, the targeting constructs for disruption of target gene also include an exogenous nucleotide sequence encoding a resistance marker protein. As described above regarding various possible types of DEL selection markers, a resistance marker conveys resistance to specific physical or chemical agents that would otherwise be toxic to a cell. The resistance marker gene is positioned between two flanking homology regions so that it integrates into the target gene following the crossover event in a manner such that the resistance marker gene is positioned for expression after integration. By imposing the selectable condition, one may isolate cells that stably express the resistance marker-encoding vector sequence from other cells that have not successfully integrated the vector sequence on the basis of viability.

The above-described use of a resistance marker does not distinguish between cells that have integrated the vector by targeted homologous recombination at the target gene locus rather than by random, non-homologous integration of vector sequence into any chromosomal position. Therefore, when using a replacement vector for homologous recombination to make the cells of the invention, it is also preferred to include a nucleotide sequence encoding a negative selectivity marker protein. As described above regarding various possible types of DEL selection markers, negative selectivity marker is a protein that when expressed is toxic to a cell. The nucleotide sequence encoding a negative selectivity marker is positioned outside of the two homology regions of the replacement vector. Given this positioning, cells will only integrate and stably express a negative selectable marker if integration occurs by random, non-homologous recombination; homologous recombination between the target gene and the two regions of homology in the targeting construct excludes the sequence encoding the negative selectable marker from integration. Thus, by imposing the negative condition, cells that have integrated the targeting vector by random, non-homologous recombination lose viability.

Vectors containing the bioluminescent markers and/or DEL selection markers (or target sequences thereof) may be introduced into a cell according to standard methods well known to those with skill in the art or those that will be apparent to them based upon the present disclosure. As those skilled in the art will appreciate, the transformation protocol chosen will depend upon, for example, the cell type and the nature of the gene of interest, and can be chosen based upon routine experimentation. Several transformation protocols are reviewed in Kaufman (1988). Methods may include electroporation, calcium-phosphate precipitation, retroviral infection, microinjection, biolistics, liposome transfection, DEAE-dextran transfection, or transferrinfection (see, e.g., Neumann et al. (1982); Potter et al. (1984); Chu et al. (1987); Thomas and Capecchi (1987); Baum et al. (1994); Biewenga et al., (1997); Zhang et al., (1993); Ray and Gage (1992); Lo (1983); Nickoloff et al. (1998); Linney et al. (1999); Zimmer and Gruss, (1989); and Robertson et al., (1986). A preferred method in the practice of the present invention for introducing foreign DNA into a yeast cell involves the use of lithium acetate/PEG, as described in Gietz and Woods (2002).

Cells to be used in the practice of the methods of the invention may be stored and cultured according to methods well known to those with skill in the art based upon the present disclosure. For example, mammalian cells may be cultured according to methods described in Bonifacino et al. (2003), Chapter 1. Yeast cells may be cultured according to general methods described in Ausubel et al. (2003), Chapter 13.

In the practice of the methods of the invention, the treatment of cells with a test agent may be employed according to methods known by those with skill in the art based upon the present disclosure. The method used will depend upon many variables, including the types of cells used, characteristics of the DEL selection marker and bioluminescent marker and characteristics of the test agents used.

In one embodiment, yeast cells (Saccharomyces cerevisiae) having a disruption of the his gene as the DEL selection marker are treated with test agents in 96 well plates for about 17 hours at about 30° C. Following treatment the cells are washed, for example, with PBS, and sonicated to assure dissociation of the cells into a single-cell suspension. The cells are then plated at an appropriate dilution (see below) onto medium lacking histidine as well as standard medium containing histidine. The histidine-lacking medium is used to determine recombination frequency. Standard medium (medium containing histidine) is used to determine the overall toxicity of the test agent.

In order to determine the optimal cell dilution for plating, the cells may be counted using a cell counting device (e.g., using a Coulter Particle Counter, Coulter Corp., Miami, Fla.). Ten fold serial dilutions are then prepared (D₀-D₅, wherein D₀ is the initial cell culture). The optimal cell dilution is such that there are sufficient cells to be able to measure: (a) the toxicity of the test agent; (b) the baseline recombination frequency of the cells (without treatment); and (c) the level of DEL recombination following treatment. For example, a preferred dilution when using S. cerevisiae cells is 1×10⁵ to 1×10⁷ cells per mL.

For high throughput detection, cells may be plated on multi-well plates (e.g., 12, 24 or 48 wells). The cells are then incubated for a sufficient time to enable revertant colonies to grow, preferably about 48 hours at about 30° C. for S. cerevisiae cells.

As those with skill in the art will appreciate, based upon the present disclosure, the bioluminescent revertant colonies may be visualized using any light detection device, for example, a Lumi-Imager® F1 photon-counting device (Roche Diagnostics, Indianapolis, Ind.) that may be used to identify colonies in multi-well plates. Other light detection devices that may be used include NightOwl (Berthold, Germany) and Kodak IS1000 (Kodak, Rochester, Md.). Furthermore, the digital image of bioluminescent colonies of cells is suitable for automated data evaluation using image analysis software (for example, Image Plus PrO™, ver. 4.1 (Media Cybernetics, Inc., Carlsbad, Calif.).

The reversion frequency may be expressed as the number of revertant cells per the total number of cells that survive treatment with the test agent. For example, for S. cerevisiae having the his- DEL selection marker the following formula may be used to calculate reversion frequency:
FR=(R×D)/(S×D′)
where:
FR=reversion frequency
R=number of revertant colonies on histidine lacking medium
S=number of colonies on standard media (containing
D=dilution factor of cells plated on histidine lacking media
D′=dilution factor of cells plated on standard media

Any statistically significant increase in the reversion frequency as compared to a control will be indicative of a test agent having potential genotoxic and/or carcinogenic properties. The determination of statistical significance is well known to those with skill in the art or will be apparent based upon the present disclosure. Preferably, the results will yield a p-value that is no more than 0.05, more preferably no more than 0.01 (Brownlee (1960)). Alternatively, the increase in reversion frequency is at least about 2-fold over the control.

As will be apparent to those with skill in the art based upon the present disclosure, the determination in a cell population of reversion frequency as compared to a control through bioluminescence requires correction for secondary effects of a test agent. For example, certain test agents that cause increased reversion frequency, may also reduce the rate of growth and/or division of cells. As a result, the number of revertant cells in untreated control cells may grow faster than those in the treated cell population such that the total bioluminescent population in the control exceeds those in the treated cells. Such secondary effects will be especially evident if bioluminescence of cells is measured en mass (e.g., by placing the cells in a liquid medium and measuring total bioluminescence).

A preferred method for correcting such secondary effects is by immobilizing populations of individual treated and control cells, e.g., using selection media which is solid or semi-solid, such that the cells form individual colonies. The reversion frequency would then be determined based upon the number of bioluminescently detectable colonies or micro-colonies.

The above-described assay methods are for illustrative purposes only. Those with skill in the art will appreciate based upon the present disclosure that a variety of assay formats may be utilized in the practice of this invention. Variations may be made based upon the types of cells, DEL selection markers, bioluminescent markers and test agents used, methods of treating and culturing cells and methods of detection of revertants.

Although the preferred use of the methods and cells of the invention is for detection of chemical mutagenic/genotoxic agents, the invention is also applicable to other agents that may cause mutagenicity/genotoxicity, for example, environmental agents such as ionizing radiation.

The disclosures of all patents, applications, publications and documents, including brochures and technical bulletins, cited herein, are hereby expressly incorporated by reference in their entirety. It is believed that one skilled in the art can, based on the present description, including the examples, drawings, and attendant claims, utilize the present invention to its fullest extent.

The following Examples are to be construed as merely illustrative of the practice of the invention and not limitative of the remainder of the disclosure in any manner whatsoever.

### EXAMPLES

### Example 1

### Preparation of the Yeast Tester Strain RS112-luc

Preparation of the pYES-GL3-GPD Plasmid.

Five μg of the plasmid, pGL3-control (Promega, Madison, Wis.), were digested with 100 units of HindIII and 100 units of XbaI (New England Biolabs, Beverly, Mas.) in presence of manufacturer-supplied buffer (20 μl total reaction volume) at 37° C. for 1 hour. The sample was then loaded onto a 0.8% agarose gel and run in TAE buffer (40 mM Tris-acetate; 2 mM Na₂EDTA×2H₂O) at 50 mV for one hour. Bands were stained using ethidium bromide (5 ng/ml for 30 minutes) and visualized on a transluminator at 2500 μW/cm². Two bands were visible, a 1.7 kb band of the luciferase gene and a 3.5 kb band containing the plasmid backbone. The 1.7 kb luciferase DNA containing band was excised as an agar gel plug and the DNA was purified from the plug using QlAquick kit (QIAGEN, Valencia Calif.).

Five pg of the expression vector, pYES6/CT (Invitrogen, Carlsbad, Calif.), were digested with HindIII and XbaI (both from (New England Biolabs) as described above and the resulting digest was separated by agarose gel electrophoresis as described above. The resulting single detectable 5.8 kb fragment band was excised and purified as described above.

The resulting luciferase and linearized pYES6/CT fragments were ligated using Rapid DNA ligation Kit (Roche Molecular Biochemicals, Indianapolis, Ind.) according to the manufacturer's protocol to make the plasmid, pYES-GL3.

The ligation mixture containing pYES-GL3 was used to transform E. coli cells (UltraMAx™ DH5α-FT™ Competent cells, Life Technologies, Rockville, Md.) according recommended protocol of the manufacturer. The transformed cells were plated on ampicillin media and a colony containing pYES-GL3 was selected.

The strong constitutive promoter for glyceraldehydes-3-phosphate dehydrogenase (GPD) (see Mumberg et al. (1995)), was prepared by digestion of p416 GPD (ATCC deposit number 87361, deposited by M. Funk) with SacI and HindIII to make a 0.7 kb DNA fragment.

The SwaI cloning site in pYES-GL3 was changed to SacI by digesting pYES-GL3 with SwaI as described above and removing of the 5′ phosphate groups by treating with calf intestinal alkaline phosphatase (New England Biolabs) at 37° C. for two hours. The resulting DNA was purified using QlAquick kit (QIAGEN), resuspended in 10 μl of sterile water and ligated to SacI linkers (New England Biolabs) using Rapid DNA ligation Kit (Roche Molecular Biochemicals) to make the pYES-GL3-Sac plasmid which was isolated from transformed E. coli cells (UltraMAx™ DH5α-FT™ Competent cells, Life Technologies). The isolated pYES-GL3-Sac was digested with SacI and HindIII to remove Pgal 1 promoter, resulting in a 0.97 kb fragment encoding the Pgal 1 promoter and a 6.5 kb fragment encoding the plasmid.

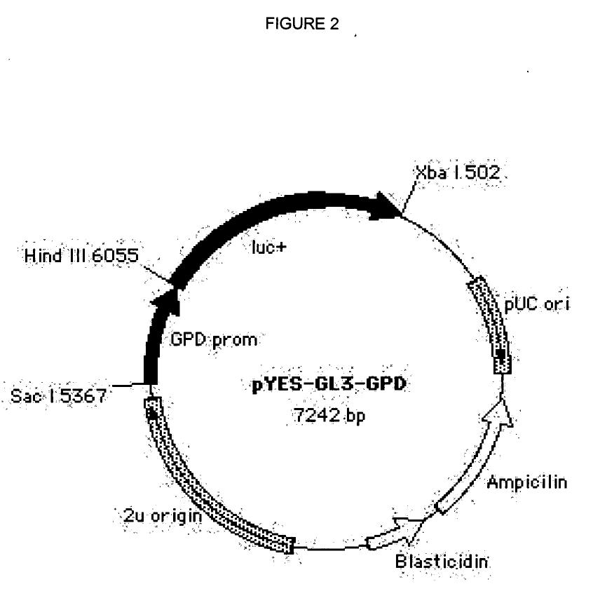

The SacI/HindIII flanked GPD promoter fragment was ligated into the pYES-GL3-Sac fragment using Rapid DNA ligation Kit (Roche Molecular Biochemicals) to make the pYES-GL3-GPD plasmid (see FIG. 2).

Preparation of the RSY112-luc Strain

Twenty-fifty microliters inoculum of the yeast strain RS112 (described in U.S. Pat. No. 4,997,757, col. 34, lines 46-64) was collected from a YPD plate (Bio101, Carlsbad, Calif.) and resuspended in one ml of sterile water. The cells were spun down using a microcentrifuge for 15 seconds and the water was removed. The resulting cell pellet was resuspended in a transformation mixture consisting of 50% PEG (240 μl), 1 M lithium acetate (36 μl), 2 mg/ml single stranded DNA (25 μl), 1 μg of the pYES-GL2-GPD plasmid (5 μl) and sterile water (45 μl) and incubated at 42° C. for 60 to 180 minutes. Following transformation, the cell suspension was spun down in microcentrifuge for 15 second and the supernatant was discarded. The resulting pellet was gently resuspended in 200-400 μl sterile water and the cells were plated on agar plates containing 50 μg/ml blasticidin (Invitrogen). Colonies of transformed blasticidin resistant yeast cells were visible after three days at 30° C. One luminescent colony in the presence of 0.4 mM luciferin (Promega) in liquid media was selected to make RS112-luc cells.

Example 1 illustrates the preparation of RS112-luc cells of this invention that may be used in the methods of the invention.

### Example 2

### Assay to Identify Genotoxic Agents Using RS112-luc Cells

An inoculum of RS112-luc cells were grown overnight (17-22 hours) at 30° C. in 50 mL of the minimum leucine deficient medium (-LEU medium) consisting of 4 g yeast nitrogen base (Bio101, Carlsbad, Calif.), 0.4 g -Leu amino acid mixture (1.8 g adenine hemisulphate, 1.2 g histidine HCl, 1.2 g uracil, all from Sigma-Aldrich Co., St. Louis, Mo.) and 12 g dextrose dissolved in 600 ml water. Prior to inoculation, the -LEU medium was sterilized by autoclaving and 4 mL of filter sterilized adenine hemisulphate (2.5 mg/mL) solution was added to replenish lost adenine due to autoclaving. The resulting culture of RS112-luc cells were spun at 32000 RPM and washed twice in PBS. The cells were then resuspended in -LEU medium at a concentration of 2×10⁶/ml and placed on ice. The cells were treated with the genotoxic methylating agent, methyl methanesulfonate (MMS, Sigma-Aldrich Co.) for 17 hrs at 30° C. in 96 well plates. Each well contained 200 μl of cell suspension plus an appropriate concentration of test compound or vehicle. After treatment, the plates were spun for 10 minutes at 3200 RPM and the supernatant was removed. The cells were then washed twice in 200 μl PBS and resuspended for five minutes using a sonicator (Branson Ultrasonic Corp., Danbury, Conn.). Cell concentration was measured using a Coulter particle counter (Coulter Corp., Miami, Fla.) to determine the most suitable dilution for plating. Dilutions of cell suspension designated Do for undiluted culture and D₁ to D₅ for serial ten-fold dilutions were prepared. Fifty μl of cell the suspension were plated on two sets of multi-well plates (e.g., 12-, 24-, or 48-well plates). The first set, for detecting revertants, contained agar medium lacking histidine consisting of 0.6 g yeast nitrogen base (Bio101, Carlsbad, Calif.), 0.7 g -his amino acid mixture (1.8 g adenine hemisulphate, 1.8 g leucine, 1.2 g uracil) (all from Sigma-Aldrich Co.), 2 g dextrose (Fisher Scientific, Fair Lawn, N.J.) and 1.7 g agar (Bacto Agar, Becton Dickinson, Sparks, Md.) dissolved in 10 ml of water. The second set, for detecting cellular viability, contained basic plus 4 medium consisting of 0.6 g yeast nitrogen base, 0.4 g +4 amino acid mixture (1.8 g adenine hemisulphate, 1.2 g histidine HCl, 1.8 g leucine 1.2 g uracil (all from Sigma-Aldrich Co.), 2 g dextrose (Fisher Scientific) and 1.7 g agar (Bacto Agar, Becton Dickinson). After plating, the cells were overlayed with 150 μl luciferin containing soft agar consisting of 5 ml of 2.4% agar in water, 5 ml PBS, 10 μl of 50 mg/ml blasticidin and 40 μl of 0.1M beetle luciferin (Promega, Madison, Wis.). The plates were then incubated for about 48 hours at 30° C. The colonies of bioluminescent revertants or surviving cells were visualized using photon-counting device Lumimager (Lumimager F1, Roche Diagnostics, Indianapolis, Ind.).

Example 2 illustrates the methods of the invention wherein RS112-luc cells of the invention are tested to determine genotoxicity of a test agent based upon the number of revertant cells based upon their level of bioluminescence.

### Deposit of RS112-luc Cells

The RS112-luc cells of this invention have been deposited under the Budapest Treaty on the International Recognition of the Deposit of Microorganisms for the Purposes of Patent Procedure with the American Type Culture Collection (ATCC) located in Manassas, Va., United States of America on Feb. 17, 2004 as patent deposit designation PTA-5822.

### Literature

Amacher, D. E. et al., “Point mutations at the thymidine kinase locus in L5178Y mouse lymphoma cells. Application to genetic toxicological testing,” Mut. Res. 64:391-406 (1979);
Ames, B. N. “The detection of chemical mutagens with enteric bacteria,” In Chemical Mutagens: Principles and Methods for Their Detection, A. Hollaender, ed., Plenum, N.Y., 1: 267-282 (1971);
Ames, B. N., “Carcinogens are mutagens: their detection and classification,” Environ. Health Perspect. 6: 115-118 (1973);
Ames, B. N. et al., “Carcinogens are mutagens: a simple test system combining liver homogenates for activation and bacteria for detection,” Proc. Natl. Acad. Sci. USA 70: 2281-2285 (1973a)(co-incubation of a carcinogen, a rat or human liver homogenate, and the bacterial tester strain on a petri plate);
Ames, B. N. et al., “An improved bacterial test system for the detection and classification of mutagens and carcinogens,” Proc. Natl. Acad. Sci. USA 70: 782-786 (1973b);
Ames, B. N. et al., “Methods for detecting carcinogens and mutagens with Salmonella/mammalian-microsome mutagenicity test,” Mutation Res. 31: 347-364 (1975);
Aubrecht, J. et al., “Carcinogens induce intrachromosomal recombination in human cells,” Carcinogenesis 16(11): 2841-6 (1995);
Ausubel, F. M. et al., Current Protocols in Molecular Biology, John Wiley & Sons, New York N.Y. (2003);
Baum, C. et al., “An optimized electroporation protocol applicable to a wide range of cell lines,” Biotechniques 17:1058-62 (1994);
Biewenga, J. E. et al., “Plasmid-mediated gene transfer in neurons using the biolistics technique,” J. Neuroscience Methods 71:67-75 (1997);
Bishop, A. J. R. and Schiesti, R. H., “Homologous recombination as a mechanism for genome rearrangement: environmental and genetic effects,” Human Molecular Genetics 9(16): 2427-2434 (2000);
Bollag, R. J, et al., “Homologous recombination in mammalian cells,” Annu. Rev. Genet. 23:199-225 (1989).
Bonifacino, J. S., et al., Current Protocols in Cell Biology, John Wiley & Sons, New York N.Y. (2003);
Bosworth, D. et al., “A forward mutation assay using ampicillin-resistance in Escherichia coli designed for investigating the mutagenicity of biological samples,” Mutagenesis 2 (6): 455468 (1987);
Bronstein, D. et al., “REVIEW: Chemilumiescent and Bioluminescent Reporter Gene Assays,” Anal. Biochem. 219:169-181 (1994);
Brooks, T. M., “The use of a streamlined bacterial mutagenicity assay, the MINISCREEN,” Mutagenesis 10(5): 447-448 (1995);
Brownlee, K. A., Statistical Theory and Methodology, Wiley, N.Y. (1960);
Brummelkamp, T. R. et al., “A system for stable expression of short interfering RNAs in mammalian cells,” Science 296: 550-553.
Chu et al., “Electroporation for the efficient transfection of mammalian cells with DNA,” Nucleic Acids Res. 15:1311-26 (1987);
Colbere-Garapin, F., Horodniceanu, F., Kourilsky, P. and Garapin, A. C., “A new dominant hybrid selective marker for higher eukaryotic cells,” J. Mol. Biol. 150: 1-14 (1981);
Cote et al (1995), “A miniaturized Ames mutagenicity assay employing bioluminescent strains of Salmonella typhimurium”, Mutation Research, 345, 137-146;
Deng, C. and Capecchi, M. R., “Reexamination of gene targeting frequency as a function of the extent of homology between the targeting vector and the target locus,” Mol. Cell. Biol. 12:3365-3371 (1992);
Evans, H. J., “Cytogenetic methods for detecting effects of chemical mutagens,” Ann. N.Y. Acad. Sci., 407:131-41 (1983);
Falck, K., et al., “Mutascreen®, an automated bacterial mutagenicity assay,” Mutation Res. 150: 119-125 (1985);
Fenech, M. and Morley, A. A., “Measurement of micronuclei in human lympocytes,” Mutat. Res., 148: 29-36 (1985);
Fire, A. et al., “Potent and specific genetic interference by double-stranded RNA in Caenorhabditis elegans,” Nature 391: 806-811.
Foster, P. L. et al., “Creation of a test Plasmid for Detecting G.C-to-T.A Transversions by Changing Serine to Arginine in the Active Site of β-Lactamase,” J. Bacteriol. 169(6): 2476-2481 (1987);
Frackman, S. et al., “Cloning, organization, and expression of the bioluminescence genes of Xenorhabdus luminescens,” J. Bacteriol. 172(10): 5767-5773 (1990);
Galli and Schiestl (1998), “Effects of DNA double-strand and single-strand breaks on intrachromosomal recombination events in cell-cycle-arrested yeast cells,” Genetics, 149, 1235-50;
Gatehouse, D. G. & Delow, G. F., “The development of a ‘Microtitre’ fluctuation test for the detection of indirect mutagens, and its use in the evaluation of mixed enzyme induction of the liver,” Mutation Res. 60: 239-252 (1979);
Gee, P. et al., “Detection and classification of mutagens: A base set of base-specific Salmonella tester strains,” Proc. Natl. Acad. Sci. USA 91: 11606-11610 (1994);
Gietz, R. D. and Woods, R. A., “Transformation of yeast by the LiAc/ss carrier dna/PEG method,” Methods in Enzymology 350: 87-96 (2002);
Grossblatt, N., Ed., “Quantitative Relationship Between Mutagenic and Carcinogenic Potencies: A Feasibility Study,” Report of the Committee on Chemical Environmental Mutagens, National Academy Press, Washington, D.C., 17-26 (1983);
Haworth, S., et al., “Salmonella mutagenicity results for 250 chemicals,” Environ. Mutagen. 5(Suppl. 1): 3-142 (1983);
Hartman, S. C., Mulligan, R. C., “Two dominant-acting selectable markers for gene transfer studies in mammalian cells,” Proc. Natl. Acad. Sci. USA 85: 8047-8051 (1988);
Hour, T-C. et al., “A new mutagenicity assay method for frameshift mutagens based on deleting or inserting a guanosine nucleotide in the β-lactamase gene,” Mutagenesis 10 (5): 433-438 (1995);
Jassim, S. A. A. et al., “In vivo Bioluminescence: A Cellular Reporter for Research and Industry,” J. Biolumin. Chemilumin. 5:115-122 (1990);
Kado, N. Y. et al., “A simple modification of the Salmonella liquid incubation assay: Increased sensitivity for detecting mutagens in human urine,” Mutat. Res. 112:25-32 (1983);
Kato, H. et al., “Automation of the Ames test,” in JEMS Abstract 31, published in Mutation Res. 334: 399 (1995);
Kaufman, R. J., Meth. Enzymology 185:537 (1988)
Lee, C-C. et al., “A reverse mutagenicity assay for alkylating agents based on a point mutation in the β-lactamase gene at the active site serine codon,” Mutagenesis 9 (5): 401-405 (1994);
Lichtenstein, P., Holm, N. V., Verkasalo, P. K., Iliadou, A., Kaprio, J., Koskenvuo, M., Pukkala, E., Skytthe, A. and Hemminki, K., “Environmental and Heritable Factors in the Causation of Cancer,” N. Engl. J. Med., 343:78-85 (2000);
Linney et al., Dev. Biol. (Orlando) 213:207-16 (1999);
Lo, C. W., “Transformation by iontophoretic microinjection of DNA: multiple integrations without tandem insertions,” Mol. Cell. Biol. 3:1803-14 (1983).
Lowy, I., Pellicer, A., Jackson, J. F., Sim, G. K., Silverstein, S., Axel, R., “Isolation of transforming DNA: cloning the hamster aprt gene,” Cell. 22: 817-23 (1980);

Maron, D. M. and Ames, B. N., “Revised methods for the Salmonella mutagenicity test,” Mutation Res. 113: 173-215 (1983);
McCann, J. et al., “Detection of carcinogens as mutagens: bacterial tester strains with R factor plasmids,” Proc. Natl. Acad. Sci. USA 72(3): 979-983 (1975);
McPherson, M. F. and Nestmann, E. R., “The SIMULTEST approach for testing mutagens in the Salmonella microtitre fluctuation assay,” Environ. Mol. Mutagen. 16: 21-25 (1990);
Monnat, R. J. et al. “Molecular structure and genetic stability of human hypoxanthine phosphoribosyltransferase (HPRT) gene duplications,”0 Genomics 13: 788-796 (1992):
Moolton, F., “Tumor chemosensitivity conferred by inserted herpes thymidine kinase genes: paradigm for a prospective cancer control strategy,” Cancer Res. 46: 5276-5281 (1986);
Mumberg, D. et al., “Yeast vectors for the controlled expression of heterologous proteins in different genetic backgrounds,” Gene 156(1): 119-22 (1995);
Neumann et al., EMBO J. 1:841-845 (1982);
Potter et al., Proc. Natl. Acad. Sci USA 81:7161-65 (1984);
Prival, M. J. and Mitchell, V. D, “Analysis of method for testing azo dyes for mutagenic activity in Salmonella typhimurium in the presence of flavin mononucleotide and hamster liver S-9,” Mutat. Res. 97:103-116 (1982);
Ray and Gage, “Gene transfer into established and primary fibroblast cell lines: comparison of transfection methods and promoters,” Biotechniques 13:598-603 (1992);
Rasmuson, B., Svahlin H., Rasmuson A., Montell I. and Olofsson H., “The use of a mutationally unstable X-chromosome in Drosophila melanogaster for mutagenicity testing,” Mutat. Res. 54(1):33-38 (1978);
Reid, T. M., Morton, K. C., Wang, C. Y., and King, C. M., “Mutagenesis of azo dyes following metabolism by different reductive/oxidative systems,” Environ. Mutagen. 6:247-259 (1984);
Robertson et al., Nature 323:445-48 (1986).
Sambrook, J., et al., Molecular Cloning: A Laboratory Manual, 2^{nd} ed., CSH Laboratory Press, Cold Spring Harbor, N.Y. (1989);
Schiestl, R. H., Igarashi, S. and Hastings, P. J., “Analysis of the Mechanism for Reversion of a Disrupted Gene,” Genetics 119:237-247 (1988);
Schiestl, R. H., “Nonmutagenic carcinogens induce intrachromosomal recombination in yeast,” Nature 337(6204): 285-288 (1989);
Scharf, D. et al. Results Probl. Cell Differ. 20:125 162 (1994).
Sommers, C. H., Mackay, W. J., Nalezny, J., Gee, P., Benjamin, M. and Farr, S. B., “Induction of DEL recombination in the yeast Saccharomyces cerevisiae using a microtiter plate format,” In Vitro and Molecular Toxicology, 11: 37-47 (1995);
Thomas, K. R. and Capecchi, M. R., “Site-directed mutagenesis by gene targeting in mouse embryo-derived stem cells,” Cell 51:503-12 (1987);
Van der Sand, S. T. et al., “Optimization of a lux gene expression in Saccharomyces cerevisiae as a tool to study gene regulation and plasmid segregation,” J. Bioluminescence and Chemiluminescence 7: 220 (1992);
Waldman, A. S. and Liskay, R. M., “Dependence of intrachromosomal recombination in mammalian cells on uninterrupted homology,” Mol. Cell. Biol. 8:5350-5357 (1988).
Waleh, N. S., et al., “Development of a toxicity test to be coupled to the Ames Salmonella assay and the method of construction of the required strains,” Mutat. Res. 97:247-256 (1982);
Walker, G. C., “Plasmid (pKM101)-mediated enhancement of repair and mutagenesis: dependence on chromsomal genes in Escherichia coli K-12, “Mol. Gen. Genet. 152(1): 93-103 (1977);
Wigler, M., Silverstein, S., Lee, L. S., Pellicer, A., Cheng, Y. and Axel, R., “Transfer of purified herpes virus thymidine kinase gene to cultured mouse cells,” Cell. 11: 223-32 (1977);
Wigler, M., Perucho, M., Kurtz, D., Dana, S., Pellicer, A., Axel, R. and Silverstein, S., “Transformation of mammalian cells with an amplifiable dominant-acting gene,” Proc. Natl. Acad. Sci. USA 77: 3567-70 (1980);
Wilcox, P. et al., “Comparison of Salmonella typhimurium TA102 with Escherichia coli WP2 tester strains,” Mutagenesis 5: 285-291 (1990);
Winson, M. K., et al., “Engineering the luxCDABE genes from Photorhabdus luminescens to provide a bioluminescent reporter for constitutive and promoter probe plasmids and mini-Tn5 constructs,” FEMS Microbiol. Lett. 163(2): 193-202 (1998);
Wolff S., “Sister chromatid exchange as a test for mutagenic carcinogens,” Ann. N. Y. Acad. Sci., 407:142-153 (1983);
Xi, L. et al., “Cloning and nucleotide sequences of lux genes and characterization of luciferase of Xenorhabdus luminescens from a human wound,” J. Bacteriol. 173: 1399-1405 (1991);
Yahagi, T. et al., “Mutagenicity of carcinogenic azo dyes and their derivatives,” Cancer Lett. 1:91-97 (1975); and
Zeiger, E., “The Salmonella mutagenicity assay for identification of presumptive carcinogens,” in the Handbook of Carcinogen Testinq (H. A. Milman and E. K. Weisburger, eds., Noyes Publishers, Park Ridge, N.J.): 83-99 (1985);
Zhang et al., “Efficient production of single-stranded DNA as long as 2 kb for sequencing of PCR-amplified DNA,” Biotechniques 15:868-72 (1993); and
Zimmer and Gruss, “Production of chimaeric mice containing embryonic stem (ES) cells carrying a homoeobox Hox 1.1 allele mutated by homologous recombination,” Nature 338:150-153 (1989).

1. A method for characterizing a test agent, comprising, treating a eukaryotic cell comprising a DEL selection marker and a bioluminescent marker with a test agent and measuring the level of bioluminescence from the cell in the presence of a suitable selection medium.

2. A method for characterizing a test agent, comprising, providing a eukaryotic cell culture comprising a DEL selection marker and a bioluminescent marker, treating a treated portion of said eukaryotic cell culture with a test agent, measuring the number of bioluminescent cells of the treated portion in the presence of a suitable selection medium, and measuring the number of bioluminescent cells of an untreated portion of the eukaryotic cell culture in the presence of said selection medium.

3. A method for characterizing a test agent, comprising, providing a eukaryotic cell culture comprising a DEL selection marker and a bioluminescent marker, treating a treated portion of said eukaryotic cell culture with a test agent, measuring the number of bioluminescent cells of the treated portion in the presence of a suitable selection medium, measuring the number of bioluminescent cells of an untreated portion of the eukaryotic cell culture in the presence of said selection medium, and characterizing the test agent according to a category selected: an agent that increases the number of bioluminescent cells of said treated portion as compared to said untreated portion in the presence of said selection medium; and an agent that does not increase the number of bioluminescent cells of said treated portion as compared to said untreated portion in the presence of said selection medium.

4. A method according to claim 3 wherein said increase in bioluminescent cells is statistically significant as compared to said untreated portion.

5. A method according to claim 3 wherein said increase in bioluminescent cells is at least two-fold as compared to said untreated portion.

6. A method according to any one of claims 1 to 5 wherein said eukaryotic cell or cell culture is derived from a cell selected from: a mammalian lymphoid cell; a human lymphoblastoid cell; a yeast cell; and a Saccharomyces cerevisiae cell.

7. A method of any one of claims 1 to 5 wherein said eukaryotic cell or cell culture is derived from Saccharomyces cerevisiae.

8. A eukaryotic cell comprising a DEL selection marker and a bioluminescent marker.

9. A cell of claim 8 derived from a cell selected from: a mammalian lymphoid cell; a human lymphoblastoid cell; a yeast cell; and a Saccharomyces cerevisiae cell.

10. A cell of claim 9 that is a Saccharomyces cerevisiae cell.

11. A cell of claim 9 of the strain RS112-luc (ATCC Deposit Designation PTA-5822).

12. A kit comprising a cell of any one of claims 8-11 and a container.

13. A kit comprising spores of a yeast cell comprising a DEL selection marker and a bioluminescent marker.

14. A kit of claim 13 wherein said yeast cell is Saccharomyces cerevisiae.

15. A kit of claim 13 wherein said yeast cell is derived from strain RS112-luc (ATCC Deposit Designation PTA-5822).
